(19) United States
(12) Patent Application Publication
(10) Pub. No.: US 2024/0090644 A1
(43) Pub. Date: Mar. 21, 2024

(54) Healing Crystal Poly Gel Nail Cover and Method of Use

(71) Applicant: Elyse James, Victorville, CA (US)

(72) Inventor: Elyse James, Victorville, CA (US)

(21) Appl. No.: 18/461,983

(22) Filed: Sep. 6, 2023

Related U.S. Application Data
(60) Provisional application No. 63/407,351, filed on Sep. 16, 2022.

(51) Int. Cl. A45D 31/00; A61K 8/04; A61Q 3/02

(52) U.S. Cl. CPC A45D 31/00; A61K 8/042; A61Q 3/02; A61K 2800/81

(57) ABSTRACT

The present invention relates to a novel gemstone nail cover and a healing crystal poly gel for providing healing effects and acupressure effects to a user. In one embodiment, the poly gel includes acrylic powder, healing crystals powder, and clear gel wherein when the poly gel is applied on a natural nail, the healing crystals powder touches the nail of the user and provides desired healing effect. In another embodiment, a gemstone nail cover is provided which is durable and harder to break. In some embodiments, a method of applying poly gel using an UV lamp or a LED lamp is described for providing a smooth putty-like look and for providing a healing and acupressure effect to a user.

### CROSS-REFERENCE TO RELATED APPLICATION

The present application claims priority to, and the benefit of, U.S. Provisional Application No. 63/407,351, which was filed on Sep. 16, 2022, and is incorporated herein by reference in its entirety.

### FIELD OF THE INVENTION

The present invention relates generally to the field of nail art and extensions. More specifically, the present invention relates to a novel semiprecious gemstone and healing crystal nail polish, semiprecious gemstone and healing crystal poly gel, semiprecious gemstone and healing crystal acrylic powder, semiprecious gemstone and healing crystal 3D gel, semiprecious gemstone and healing crystal gel hardener, semiprecious gemstone and healing crystal gel builder, semiprecious gemstone and healing crystal gel nail polish, semiprecious gemstone and healing crystal chrome powder, semiprecious gemstone and healing crystal soft gel, semiprecious gemstone and healing crystal sculpting gel, semiprecious gemstone and healing crystal gel, semiprecious gemstone and healing crystal nail polish lacquer, semiprecious gemstone and healing crystal nail cover, acupressure nail cover, semiprecious gemstone and healing crystal press on nails, semiprecious gemstone and healing crystal 3D nail art charm, semiprecious gemstone and healing crystal nail art brush for bringing healing crystals to the nail industry in need of versatility and options. The nail polish, poly gel, and/or acrylic powder have healing stones included therein and can be applied directly on a natural or artificial nail. Nail covers are made of gemstones, are durable, and harder to break while providing improvement of chakras and balancing of energies. Accordingly, the present disclosure makes specific reference thereto. Nonetheless, it is to be appreciated that aspects of the present invention are also equally applicable to other like applications, devices, and methods of manufacture.

### BACKGROUND

By way of background, healing crystals have been used for thousands of years promoting positive energy and wellness. Today, they are an established form of alternative medicine. An established practice, crystal healing is a pseudoscientific alternative-medicine practice that uses semiprecious stones and crystals such as quartz, agate, amethyst and many more. Many individuals believe in acupressure and it helps individuals in relieving stress and anxiety, improving sleep, relaxing muscles and joints, minimizing headaches and migraines, and more. However, healing crystals and different means of acupressure are not available to the public easily and cost-efficiently. Also, there is no way for individuals to use the gemstones and more such that such stones always touch skin or body of a user.

Nail art, nail extensions, and nail covers are commonly used by women for increasing beauty of their nails. Currently available nail extension techniques do not provide a means of adding gemstones, healing crystals, and more to nail extensions. Conventional poly gel is used in nail salons; however they do not include healing stones in any of the processes. It would be desirable for individuals, especially women, to have nail extensions and poly gel that include healing stones for providing an effective healing effect.

A problem with conventional nail extensions is that they are extremely soft and brittle. Nail extensions may break easily while doing household work and more and as a result, women want improved nails that are more durable and harder to break.

Therefore, there exists a long-felt need in the art for a way that can provide a convenient method to make healing crystals accessible and fashionable. There is also a long-felt need in the art for acupressure fingernails and toenails, gel polish, and nail cover. Additionally, there is a long-felt need in the art for applying any gemstone as an artificial nail for creating healing effects. Moreover, there is a long-felt need in the art for improved nail extensions that are made of stone and much harder to break. Further, there is a long-felt need in the art for a method that brings healing crystals to market with a variety of options. Furthermore, there is a long-felt need in the art for a poly gel that includes healing stone in crushed or powder form for allowing the powder to be in contact with a human body. Finally, there is a long-felt need in the art for nail extensions and covers that give users options for a nail made of stone which is more durable and harder to break.

The subject matter disclosed and claimed herein, in one embodiment thereof, comprises a healing crystal poly gel for application on natural nails or artificial nails (i.e., press-on nails or nail covers) of a user. The healing crystal poly gel further comprising of a crushed healing stones powder, and a clear poly gel for mixing the healing stones poly gel. The healing stones powder can be made by crushing one or more of amethyst, citrine, rose quartz, clear quartz, tiger eye, jasper, fluorite, lapis lazuli, sodalite, malachite, agate, and more. The poly gel can also be applied to nail tips or applied on a natural nail for attaching the powder or poly gel to the natural nail for creating a healing effect and balancing chakras and energies of the user. The healing crystal nail polish can be polished on natural nails or nail extensions. The healing crystal powder is mixed with acrylic powder and can be applied to natural nails or to artificial nails (i.e., press-on nails or nail covers) in an application process.

In this manner, the healing crystal poly gel and associated nail extension of the present invention accomplish all of the forgoing objectives and provide users with acupressure and healing gemstones fingernails and toenails, gel polish, and nail cover. The invention offers a convenient method to make healing crystals accessible and fashionable and provides durable and harder nail covers. The gemstone nail extensions and poly gel make accessibility of healing stones easy, cost-effective, stylish, and enable users to wear the nails as a way to heal, protect, and display their unique style.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a healing crystal poly gel for application on natural nails of a user. The healing crystal poly gel and the healing crystal nail polish further comprising of a crushed healing stones powder and a clear gel for mixing the healing stones powder. The healing stones powder can be made by crushing one or more of amethyst, citrine, rose quartz, clear quartz, tiger eye, jasper, fluorite, lapis lazuli, sodalite, malachite, agate, black tourmaline, smoky quartz, aventurine, carnelian, emerald, rainbow aura quartz, calcite, obsidian, blue apatite, chariot, and more.

The subject matter disclosed and claimed herein, in one embodiment thereof, is a method of applying crushed healing stones powder for balancing energy of a user. The method includes the steps of providing a healing crystals poly gel, the poly gel is in a gel form and includes crushed healing stones powder, and a clear gel; applying healing crystal poly gel on a natural nail or artificial nail; curing and drying the poly gel in UV light or LED light; filing the excessive poly gel material from the natural nail; buffing the nail; and, applying a clear polish on the poly gel, wherein the crushed healing stones powder embedded in the poly gel directly touches the natural nail of the user upon application of the poly gel.

In another aspect, the present invention provides a method of wearing nails as a way to heal, protect, and display unique styles. The method comprises the steps of applying crushed gemstones on a natural or an artificial press on nail; applying healing crystal poly gel on the top of the natural nail or artificial press on nail covered in the crushed gemstones; curing the nail in UV light to dry the poly gel; buffing the natural nail or artificial press on nail; applying gel nail polish by brushing nail polish on the natural nail or artificial press on nail; and, curing under UV light until dry.

In yet another embodiment, a chakra healing nail cover is disclosed. The chakra healing nail cover includes crushed chakra healing gemstones, wherein the cover may improve any of the seven chakras of the human body for improving spirituality and emotional balance.

In yet another embodiment, the poly gel may contain any healing gemstone powder.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a healing crystal nail extension of the present invention in accordance with the disclosed architecture;

FIG. 2 illustrates a block diagram showing the components in forming the healing crystals poly gel of one embodiment of the present invention in accordance with the disclosed architecture;

FIG. 3 illustrates a flow diagram depicting a process of using the healing crystal poly gel of the present invention in accordance with the disclosed architecture;

FIG. 4 illustrates a flow diagram depicting another process of applying crushed healing gemstones on nails of a user in accordance with the disclosed architecture; and

FIG. 5 illustrates a perspective view of a user wearing acupressure toenail covers of one embodiment of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It can be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments can be combined.

As noted above, there is a long-felt need in the art for a way that can provide a convenient method to make healing crystals accessible and fashionable. There is also a long-felt need in the art for acupressure fingernails and toenails, gel polish, and nail cover. Additionally, there is a long-felt need in the art for applying any gemstone as an artificial nail for creating healing effects. Moreover, there is a long-felt need in the art for improved nail extensions that are made of stone and much harder to break. Further, there is a long-felt need in the art for a method that brings healing crystals to market with a variety of options. Furthermore, there is a long-felt need in the art for a poly gel that includes healing stone crush or powder for allowing the powder to be in contact with a human body. Finally, there is a long-felt need in the art for nail extensions and covers that give users options for a nail made of stone which is more durable and harder to break.

The present invention, in one exemplary embodiment, is a method of applying crushed healing stones powder for balancing energy of a user. The method includes the steps of providing a healing crystals poly gel, the poly gel is in a gel form and includes crushed healing stones powder, and a clear gel; applying healing crystal poly gel on a natural nail; curing and drying the poly gel in UV light or LED light; filing the excessive poly gel material from the natural nail; and, applying a clear polish on the poly gel, wherein the crushed healing stones powder embedded in the poly gel directly touches the natural nail of the user upon application of the poly gel.

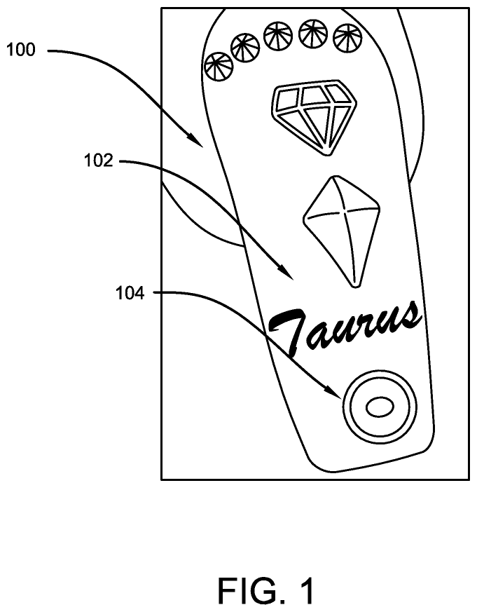

Referring initially to the drawings, FIG. 1 illustrates a perspective view of one potential embodiment of the healing crystal nail extension of the present invention in accordance with the disclosed architecture. The healing crystal nail extension 100 of the present invention is designed to enable users to wear, mount, or place artificial nail extensions onto natural nails as a way to heal, protect, and display their unique style. The nail extension 100 is easy to use and can be deployed by both salon technicians and individuals at home. The nail extension 100 in the present embodiment has crushed healing stone crystals 102 embedded therein for providing healing power and includes one or more solid healing stones 104 affixed thereon.

The nail extension or cover 100 functions like a conventional nail extension or cover to enhance the look of a natural nail but provides additional utility of providing healing, protecting the wearer from negative energies and emotions, and more. The nail cover 100 is lightweight and is more durable and harder to break than conventional nail extensions. The nail cover 100 may come in different sizes, shapes, and colors and may embed any type of healing stone as required by a user. The healing crystals may include one or more of amethyst, citrine, rose quartz, clear quartz, gold, diamond, tiger eye, jasper, fluorite, lapis lazuli, sodalite, malachite, agate, black tourmaline, smoky quartz, aventurine, carnelian, emerald, rainbow aura quartz, calcite, obsidian, blue apatite, chariot, and more.

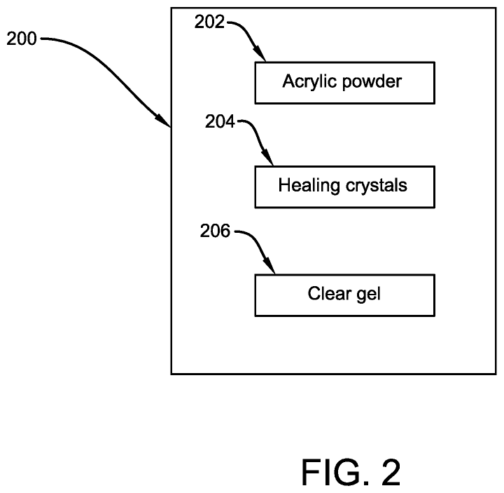

It should be noted that in the present embodiment, a healing stone nail cover is disclosed, but the invention encompasses gemstones healing crystal poly gel as well, as described in FIG. 2.

FIG. 2 illustrates a block diagram showing the components in forming the healing crystals poly gel of one embodiment of the present invention in accordance with the disclosed architecture. The present invention also provides a healing crystal poly or polymeric gel 200 for applying a gemstone as an artificial nail in order to make healing crystals accessible and fashionable. The poly or polymeric gel 200 is applied as an overlay on the natural nail of a user such that the healing crystals provide healing power for emotional and spiritual balance of the user. The poly or polymeric gel 200 includes acrylic powder 202, which is formed by crushing acrylic with a uniform thickness. The acrylic powder 202 is used for providing acrylic strength to the poly or polymeric gel 200 such that poly or polymeric gel 200 remains applied on a natural nail for a considerable amount of time.

Poly or polymeric gel 200 includes healing crystals 204, which are in crushed form of a healing gemstone. Crystals of any gemstone preferred by a user can be included in the poly or polymeric gel 200 to synergize, for example the zodiac sign of the user to bring balance to an individual's life and mind. For providing a gel texture to the poly or polymeric gel 200, a clear gel 206 is used. The clear gel 206 gives a gel texture to the poly or polymeric gel 200 and allows both the acrylic powder 202 and the healing crystals 204 to uniformly mix and provides a lightweight texture.

Healing crystals poly or polymeric gel 200 when applied on a nail is more flexible than acrylic and is stronger than hard gel. The poly or polymeric gel 200 is lightweight and has a putty-like consistency. Advantageously, oligomers from clear gel 206 and polymers from acrylic powder 202 mix well with the healing crystals 204.

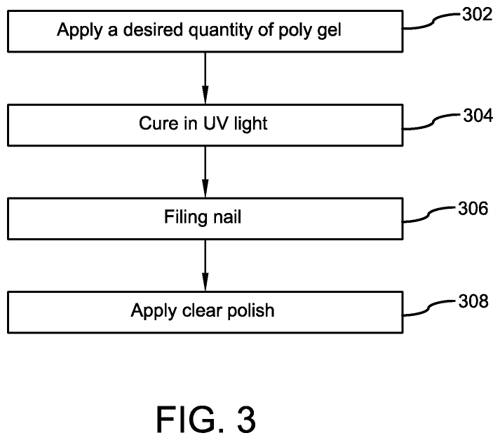

FIG. 3 illustrates a flow diagram depicting a process of using the healing crystal poly gel of the present invention in accordance with the disclosed architecture. The poly or polymeric gel 200 can be applied on any natural nail for balancing energies of a user while providing the natural nail with a coating or covering which is more durable and harder to break. For applying the poly or polymeric gel 200, a desired quantity of the healing crystal poly or polymeric gel 200 is taken from a tube such as a toothpaste tube (Step 302). An artificial color can also be provided to the poly or polymeric gel 200 as per desire of users of the product.

Next, the applied poly gel is cured under an UV light for about 2 minutes or for about 1 minute under an LED lamp (Step 304). For removing any excessive poly gel from the natural nail, filing of the dry poly gel is performed using a conventional nail filer device (Step 306). Finally, for smoothness and a putty-like feel, a clear polish is applied on the poly gel by a user (Step 308). It should be noted that the poly gel of the present invention enables users to wear the nails as a way to heal, protect, and display their unique style and offers a convenient method to make healing crystals accessible and fashionable.

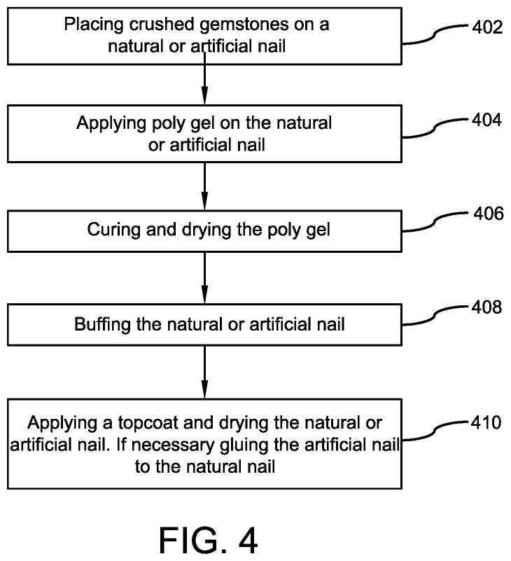

FIG. 4 illustrates a flow diagram depicting another process of applying crushed healing gemstones on nails of a user in accordance with the disclosed architecture. In the present embodiment, initially, a plurality of crushed gemstones is placed on a natural nail or an artificial press on plastic nail (Step 402). The crystals/gems/embellishments can be of any type and characteristics as per desires of individuals. Thereafter, a clear poly gel that does not include any crushed healing stones is applied on the top of the crushed gemstones (Step 404).

Thereafter, the nail is kept under a UV lamp for curing and drying the poly gel allowing a direct and stable contact of the gemstone with the nail of the user (Step 406). The nail is then buffed (Step 408). A clear topcoat can be applied to the nail and dried again for two minutes; and finally, if necessary, the artificial nail can be glued to the natural nail for an effective nail enhancement while providing healing power to a user (Step 410). In an alternative method, a nail art brush can be dipped in a monomer or poly gel solution and then dipped in a healing crystal acrylic powder. The solution and acrylic powder can then be applied or brushed onto a natural nail or artificial nail tip. Thereafter, the nail or nail tip can be air dried (or cured under UV light), buffed, and clear coated. It is to be appreciated that the monomer or poly gel solution and the healing crystal acrylic powder can be pre-mixed.

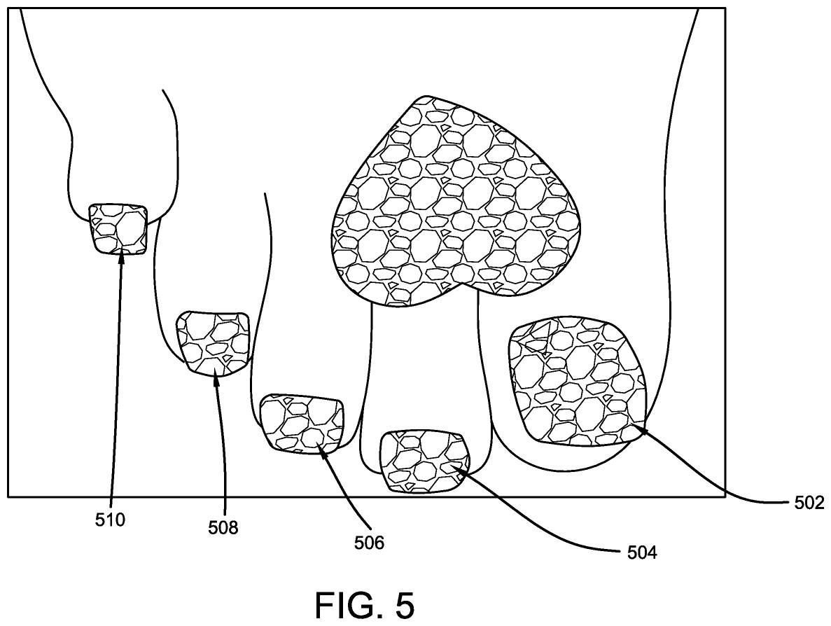

FIG. 5 illustrates a perspective view of a user wearing acupressure toenail covers of one embodiment of the present invention in accordance with the disclosed architecture. In the present embodiment, the invention offers a plurality of different types of acupressure nail covers configured to heal a user in a non-invasive manner while beautifying the natural nails of a user. As illustrated, a first nail cover 502 is related to crown chakra and improves spiritual connection and transformation. The first nail cover 502 can be from one of amethyst, clear quartz, selenite, lepidolite, howlite, labradorite, sugilite, white agate, fluorite, lapis lazuli, moonstone, charoite, and white calcite. The first nail cover 502 helps to clear blockages from the crown chakra and encourage to reach enlightenment.

The second nail cover 504 and the third nail cover 506 are related to third eye chakra or sinus chakra. The covers 504, 506 are configured to improve perception, awareness, and spiritual communication. The fourth nail cover 508 and the fifth nail cover 510 are ear chakras and can be made from amethyst, lapis lazuli, and quartzite.

It should be noted that the nail covers can be designed as chakra stones that activate or amplify the energy of the chakras and are available in different sizes and shapes to meet requirements of different users.

The poly gel and various nail covers of the present invention can be used to apply full set nails gel manicure, gel pedicures, as well as press-on fingernails, toenails, and nail tips for acrylic application for offering a convenient method to make healing crystals accessible and fashionable. The healing crystal poly gel can be used with any nail cover and can enhance healing powers for a user.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “healing crystal nail extension”, “nail extension”, and “nail cover” are interchangeable and refer to the healing crystal nail extension 100 of the present invention. Similarly, as used herein “poly gel”, “polymeric gel”, “healing crystals poly gel”, and “poly gel formula” are interchangeable and refer to the healing crystals poly (or polymeric) gel 200 of the present invention.

Other terms used herein are also interchangeable with the following: healing crystal/semi precious stone 3D gel, healing crystal/semi precious stone acrylic powder product, healing crystal/semi precious stone gel hardener product, healing crystal/semi precious stone gel builder product, healing crystal semi precious stone poly gel product, healing crystal gel nail polish product, and healing crystal nail polish product.

Notwithstanding the forgoing, the healing crystal nail extension 100 and the healing crystals poly or polymeric gel 200 of the present invention can be of any suitable size and configuration as is known in the art without affecting the overall concept of the invention, provided that it accomplishes the above-stated objectives. One of ordinary skill in the art will appreciate that the healing crystal nail extension 100 and the healing crystals poly or polymeric gel 200 as shown in the FIGS. are for illustrative purposes only, and that many other sizes and shapes of the healing crystal nail extension 100 and the healing crystals poly or polymeric gel 200 are well within the scope of the present disclosure. Although the dimensions of the healing crystal nail extension 100 and the healing crystals poly or polymeric gel 200 are important design parameters for user convenience, the healing crystal nail extension 100 and the healing crystals poly or polymeric gel 200 can be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A healing crystal nail extension comprising:
a plurality of healing crystal nail extensions for placement onto natural nails of a user;
wherein each of said plurality of healing crystal nail extensions comprising a cured mixture of acrylic powder, healing crystals, and gel;
wherein said healing crystals are crushed healing stone crystals embedded in said cured mixture; and
further wherein said crushed healing stone crystals selected from a group consisting of one or more of an amethyst, a citrine, a rose quartz, a clear quartz, a tiger eye, a jasper, a fluorite, a lapis, a lazuli, a sodalite, a malachite, an agate, a black tourmaline, a smoky quartz, an aventurine, a carnelian, an emerald, a rainbow aura quartz, a calcite, an obsidian, a blue apatite, and a chariot.

2. The healing crystal nail extension of claim 1 further comprising at least one solid healing stone affixed to said cured mixture.

3. The healing crystal nail extension of claim 2, wherein said at least one solid healing stone is a gemstone.

4. The healing crystal nail extension of claim 3, wherein said gel is a clear poly gel.

5. A method of forming a healing nail covering, the method comprising the steps of:
applying crushed healing crystals to nails of a user;
applying a poly gel over said crushed healing crystals and said nails of the user;
placing said nails under an UV light; and
curing said poly gel under said UV light, wherein said crushed healing crystals are crushed healing stone crystals, and further wherein said crushed healing stone crystals selected from a group consisting of one or more of an amethyst, a citrine, a rose quartz, a clear quartz, a tiger eye, a jasper, a fluorite, a lapis, a lazuli, a sodalite, a malachite, an agate, a black tourmaline, a smoky quartz, an aventurine, a carnelian, an emerald, a rainbow aura quartz, a calcite, an obsidian, a blue apatite, and a chariot.

6. The method of forming a healing nail covering of claim 5 wherein said nails are selected from natural nails and artificial nails.

7. The method of forming a healing nail covering of claim 6 further comprising a step of buffing said nails.

8. The method of forming a healing nail covering of claim 7 further comprising a step of applying a clear topcoat over said nails and said cured said poly gel.

9. The method of forming a healing nail covering of claim 8 further comprising a step of drying said clear topcoat.

10. The method of forming a healing nail covering of claim 9, wherein the healing nail covering is a coating on the natural nails of the user.

11. The method of forming a healing nail covering of claim 10, wherein said poly gel is a clear poly gel.

12. The method of forming a healing nail covering of claim 11, wherein said clear poly gel having oligomers.

13. The method of forming a healing nail covering of claim 12, wherein said acrylic powder having polymers.

14. A method of applying a healing nail covering, the method comprising the steps of:
mixing a composition of acrylic powder, healing crystals, and a gel to form a healing nail poly gel;
applying said healing nail poly gel to natural nails of a user;
placing the natural nails of the user under an UV light; and
curing said healing nail poly gel under said UV light, wherein said healing crystals are crushed healing stone crystals, and further wherein said crushed healing stone crystals selected from a group consisting of one or more of an amethyst, a citrine, a rose quartz, a clear quartz, a tiger eye, a jasper, a fluorite, a lapis, a lazuli, a sodalite, a gold, a diamond, a malachite, an agate, a black tourmaline, a smoky quartz, an aventurine, a carnelian, an emerald, a rainbow aura quartz, a calcite, an obsidian, a blue apatite, and a chariot.

15. The method of claim 14 further comprising a step of filing excessive said cured healing nail poly gel from the natural nails of the user.

16. The method of claim 15 further comprising a step of applying a clear polish to said cured healing nail poly gel.

17. The method of claim 16, wherein said applying said healing nail poly gel is a brushed overlay on the natural nails of the user.

18. The method of claim 17 further comprising a step of applying a gemstone to said healing nail poly gel.

19. The method of claim 17, wherein said acrylic powder having a uniform thickness of crushed said acrylic powder.

20. The method of claim 19, wherein said gel is a clear gel.
